(19) United States
(12) Patent Application Publication
(10) Pub. No.: US 2009/0149462 A1
(43) Pub. Date: Jun. 11, 2009

(54) PROCESS FOR PURIFICATION OF APREPITANT

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Thungathurthy Srinivasa Rao, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO DRUGS LIMITED, Hyderabad, (IN)

(21) Appl. No.: 11/915,864

(22) PCT Filed: Aug. 28, 2006
(86) PCT No.: PCT/IN2006/000312

§ 371 Date: Nov. 29, 2007

(51) Int. Cl. A61K 31/5377; C07D 413/06

(52) U.S. Cl. 514/236.2; 544/132

(57) ABSTRACT

The present invention relates to a process for obtaining pure aprepitant substantially free of undesired diastereomeric isomer, namely 5-[2(S)-[1(RS)-[3,5-bis(trifluoromethyl)-phenyl)ethoxy]-3-(S)-(4-fluorophenyl)-morpholin-4-yl-methyl]-3,4-dihydro-2H-1,2,4-triazol-3-one. The present invention further provides an improved process for preparation of aprepitant crystalline form II. The present invention also relates to novel amorphous form of aprepitant, a process for its preparation and to a pharmaceutical composition comprising it. The present invention further relates to aprepitant having a mean particle size of less than about 11.5 microns, a process for its preparation and to a pharmaceutical composition comprising it. Thus, for example, aprepitant having a content of diastereomeric impurity of 1.1% is dissolved in ethyl acetate at 70° C., the solution is concentrated to half the initial volume by distilling off ethyl acetate, and the resulting solid is collected at 0-5° C. to give pure aprepitant substantially free of its diastereomeric impurity.

### FIELD OF THE INVENTION

The present invention relates to a process for obtaining pure aprepitant substantially free of undesired diastereomeric isomer, namely 5-[2(S)-[1(RS)-[3,5-bis(trifluoromethyl)-phenyl)ethoxy]-3-(S)-(4-fluorophenyl)-morpholin-4-yl-methyl]-3,4-dihydro-2H-1,2,4-triazol-3-one. The present invention further provides an improved process for preparation of aprepitant crystalline form II. The present invention also relates to a novel amorphous form of aprepitant, process for its preparation and to a pharmaceutical composition comprising it. The present invention also provides aprepitant particles that have reduced particle size.

### BACKGROUND OF THE INVENTION

PCT Publication No. WO 95/16679 disclosed certain morpholine and thiomorpholine compounds as substance P antagonists, processes for their production and use thereof. Among them aprepitant, chemically 5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one is a tachykinin receptor antagonist useful in the treatment or prevention of disorders of the central nervous system, inflammatory diseases, pain or migraine, asthma, and emesis. Aprepitant is represented by the following structure:

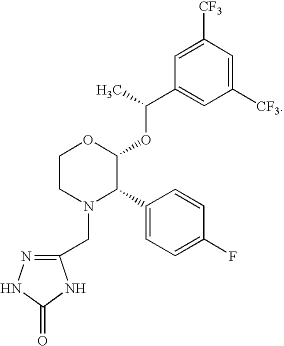

The PCT Publication No. WO 95/16679 described a process for the preparation of aprepitant, wherein the solution of 2-(R)-(1-(R)-(3,5-bis(trifluoromethyl)phenyl)ethoxy)-3-(S)-(4-fluorophenyl)morpholine, N-methyl carboxy-2-chloro-acetamidrazone, and N,N-diisopropylethylamine in acetonitrile was stirred at room temperature, the reaction mass was concentrated, the resulting residue was partitioned between methylene chloride and water, the resulting organic layer is concentrated and then subjected to flash chromatography on silica gel using 50:1:0.1 methylene chloride/methanol/ammonium hydroxide as the eluant to afford aprepitant. The publication WO 95/16679 makes no reference to the existence of specific polymorphic forms of aprepitant.

PCT publication No. WO 99/01444 described a polymorphic form of aprepitant designated as form I, characterized by an x-ray powder diffraction pattern having peaks expressed as 2θ at about 12.0, 15.3, 16.6, 17.0, 17.6, 19.4, 20.0, 21.9, 23.6, 23.8, and 24.8 degrees, process for preparing it and a pharmaceutical composition comprising it.

According to the PCT publication No. WO 99/01444, aprepitant form I can be prepared, either by (i) equilibrating aprepitant form II in a solvent which is selected from the group consisting of ethanol, 2-propanol, acetonitrile and isopropyl alcohol or (ii) heating a sample of aprepitant of optional morphological composition to a temperature range of 213 to 230° C. and then returning the sample to ambient temperature or (iii) suspending aprepitant of optional morphological composition in solution of methanol/water, adding seed crystals of aprepitant form I, stirring the resultant mixture at about 0-5° C. for a period sufficient to result in the formation of aprepitant form I and collecting the resultant aprepitant form I.

The PCT publication No. WO 99/01444 further described that the synthetic procedure described and exemplified in the PCT publication No. WO 95/16679 produces the aprepitant crystalline form designated herein as form Ii, characterized by an X-ray powder diffraction pattern having peaks expressed as 2θ at about 12.6, 16.7, 17.1, 17.2, 18.0, 20.1, 20.6, 21.1, 22.8, 23.9, and 24.8 degrees.

The PCT publication No. WO 99/01444 further described a process for the preparation of aprepitant form II, where in the solution containing crude aprepitant and methanol is subjected to carbon treatment, the resulting filtrate is cooled down to room temperature, water is added drop wise, and after being stirred at room temperature for 2 hours, the suspension is filtered to give aprepitant form II.

Various processes for preparation of aprepitant and related compounds were described, for example, in PCT publication Nos. WO 99/65900, WO 01/96315 A1 and WO 03/089429 A1.

Aprepitant obtained by the process described in the art is not satisfactory from purity point of view. The diastereomeric impurity, namely 5-[2(S)-[1(RS)-[3,5-bis(trifluoromethyl)-phenyl)ethoxy]-3-(S)-(4-fluorophenyl)-morpholin-4-yl-methyl]-3,4-dihydro-2H-1,2,4-triazol-3-one is main concern and aprepitant obtained by the prior art is contaminated with this diastereomeric impurity. Since there is a need for a process for obtaining pure aprepitant that can be used in pharmaceutical preparation.

Extensive experimentation is carried out by the present inventors to find the way to eliminate this diastereomeric impurity. As a result, it has been found that when aprepitant is crystallized from a solution of aprepitant contaminated with the diastereomeric impurity in ethyl acetate and the pure aprepitant is obtained. According to the novel process, no chromatographic separations are required for isolating pure aprepitant substantially free from diastereomeric impurity there by increasing the productivity.

One object of the present invention is to provide a commercially viable process for purification of aprepitant.

Another object of the present invention is to provide a novel process for the preparation of aprepitant crystalline form II.

The processes described in the prior art produce aprepitant having the mean particle size of about 12 microns.

It is known that, particle size can affect the dissolution properties of a drug product. Particle size reduction may be tried in order to increase dissolution characteristics of aprepitant. Particle size reduction increases the surface area of the solid phase that is in contact with the liquid medium.

Particle size also can affect how freely crystals or a powdered form of a drug will flow past each other, which has consequences in the production process of pharmaceutical products containing the drug.

In view of the foregoing, there is a need in the medical arts for aprepitant with a small particle size and improved bioavailability.

Another object of the present invention is to provide aprepitant having mean particle size of less than about 11.5 microns, process for preparing it and a pharmaceutical composition comprising it.

The processes described in the prior art produce crystalline aprepitant. It is well known that pharmaceutical products in amorphous form usually have better dissolution characteristics than when they are in crystalline form. The existence of amorphous form of aprepitant has now been discovered. The novel amorphous aprepitant is found to have better dissolution rate than the known crystalline aprepitant. So, the novel form is suitable for pharmaceutical preparations.

Another object of the present invention is to provide a novel amorphous of aprepitant, process for preparing it and a pharmaceutical composition comprising it.

### DETAILED DESCRIPTION OF THE INVENTION

According to one aspect of the present invention there is provided a process for preparing crystalline form II of aprepitant which comprises:

a) distilling off the solvent from a solution of aprepitant in a solvent selected from methanol, ethanol, isopropylalcohol and tert-butyl alcohol at least until precipitation of aprepitant occurs;
b) separating the solid aprepitant, if necessary;
c) slurrying the solid aprepitant in water; and
d) separting crystalline form II of aprepitant from the contents.

The distillation of the solvent may be carried out at atmospheric pressure or at reduced pressure. The distillation of the solvent may be carried out just until precipitation of aprepitant start forming or the distillation may be carried out until substantial precipitation occurs. The distillation may also preferably be carried out until the solvent is almost completely distilled off.

The separation of the precipitated solid aprepitant may be carried out by the methods known in the art such as filtration or centrifugation.

The solid collected is slurried in water. The temperature at which slurrying is done is not critical and the slurrying may conveniently be carried out at about 20° C. to 80° C.

The crystalline form II of aprepitant is collected from the slurry by conventional methods such as filtration or centrifugation.

The solution of aprepitant used in step (a) may be obtained by dissolving aprepitant in the solvent. The solution of aprepitant obtained as part of the synthesis of aprepitant may also be used in step (a).

The process described in the art does not yield aprepitant in the desired purity. The diastereomeric impurity, namely 5-[2(S)-[1 (RS)-[3,5-bis(trifluoromethyl)-phenyl)ethoxy]-3-(S)-(4-fluorophenyl)-morpholin-4-yl-methyl]-3,4-dihydro-2H-1,2,4-triazol-3-one, is main concern and aprepitant obtained by the prior art is contaminated with this diastereomeric impurity. Extensive experimentation is carried out to find the way to eliminate this impurity. It has been found that when aprepitant substantially free of diastereomeric impurity is crystallized from a solution of aprepitant contaminated with the diastereomeric impurity in ethyl acetate and the pure aprepitant is obtained.

The term “aprepitant substantially free of diastereomeric impurity” refers to the aprepitant containing the content of diastereomeric impurity in less than about 0.1% by weight, preferably less than about 0.05% by weight and still more preferably containing no diastereomeric impurity.

According to another aspect of the present invention, there is provided a process for purification of aprepitant which process comprises crystallizing aprepitant from a solution of crude aprepitant in ethyl acetate.

The crude aprepitant may be dissolved, if necessary, at elevated temperature. The crystallization may be initiated by any conventional methods usually known in the art such as cooling, seeding, partial removal of the solvent from the solution, by adding an anti-solvent to the solution or a combination thereof.

According to another aspect of the present invention, there is provided aprepitant having mean particle size of less than about 11.5 microns, preferably between 2-10 microns and more preferably between 3-8 microns.

According to another aspect of the present invention, there is provided a process for obtaining aprepitant having mean particle size of less than about 11.5 microns, which process comprises crystallizing aprepitant having mean particle size of less than about 11.5 microns from a solution of aprepitant in ethyl acetate.

The aprepitant may be dissolved, if necessary, at elevated temperature. The crystallization may be initiated by any conventional methods usually known in the art such as cooling, seeding, partial removal of the solvent from the solution, by adding an anti-solvent to the solution or a combination thereof.

According to another aspect of the present invention, there is provided a novel amorphous aprepitant. The amorphous aprepitant is characterized by having broad X-ray diffraction spectrum as in FIG. 1.

According to another aspect of the present invention, a process is provided for preparation of amorphous aprepitant. Amorphous aprepitant is prepared by dissolving aprepitant an alcoholic solvent, a ketonic solvent or an ester solvent, and then removing the solvent from the solution by spray drying or freeze drying.

The alcoholic solvent is selected from the group consisting of methanol, ethanol, isopropyl alcohol, tert-butylalcohol and n-butyl alcohol. The ketonic solvent is selected from the group consisting of acetone, diethyl ketone, methyl ethyl ketone, methyl isobutyl ketone and methyl propyl ketone. The ester solvent is selected from ethyl acetate, methyl acetate and isobutyl acetate. A mixture of two or more of these solvents may also be used. The preferable alcoholic solvent is methanol.

The solvent may preferable be removed from the solution by spray drying.

The “crude aprepitant” refers to aprepitant containing the content of diastereomeric impurity in about 0.1% or above by weight, preferably above 0.4% by weight and more preferably above 1.0% by weight.

According to another aspect of the present invention, there is provided a pharmaceutical composition comprising amorphous aprepitant and a pharmaceutically acceptable excipient.

Preferable pharmaceutical composition of amorphous aprepitant is a solid oral dosage form.

According to another aspect of the present invention, there is provided a pharmaceutical composition comprising aprepitant having mean particle size of less than about 11.5 microns and a pharmaceutically acceptable excipient.

Preferable pharmaceutical composition of aprepitant having mean particle size of less than about 11.5 microns is a solid oral dosage form.

### BRIEF DESCRIPTION OF THE DRAWING

FIG. 1 is X-ray powder diffraction spectrum of amorphous aprepitant.

X-ray powder diffraction spectrum was measured on a bruker axs D8 advance X-ray powder diffractometer having a copper-Kα radiation. Approximately 1 gm of sample was gently flattered on a sample holder and scanned from 2 to 50 degrees two-theta, at 0.03 degrees to theta per step and a step of 0.5 seconds. The sample was simply placed on the sample holder. The sample was rotated at 30 rpm at a voltage 40 KV and current 35 mA.

The invention will now be further described by the following examples, which are illustrative rather than limiting.

### Reference Example 1

Step-I: (2R,3S)-4-Benzyl-3-(4-fluorophenyl)morpholinyl-3,5-bis(trifluoromethyl)benzoate

(3S)-4-Benzyl-3-(4-fluorophenyl)-2-morpholinone (100 gm) is stirred with tetrahydrofuran (1.38 Lt) under N₂ atmosphere at 25-30° C., cooled to −80° C. to −70° C. with dry ice, L-selectride is slowly added to the mass for 1 hour at −80° C. to −70° C. and then stirred for 1 hour at −8° C. to −70° C. To the reaction mass added 3,5-bis(trifluoromethyl)benzoyl chloride (140 ml) slowly for 50 minutes at −80° C. to −70° C., stirred for 1 hour at 80° C. to −70° C. and then quenched into the mixture of acetic acid (9 ml) and tetrahydrofuran (35 ml) at 25-30° C. under N₂ atmosphere. The resulting mass is stirred for over night at 25-30° C., distilled off the solvent completely under vacuum at 50° C., and then n-hexane (1.55 Lt) and water (750 ml) are added. The contents are stirred for 20-30 minutes, separate the layers and the resulting organic layer is washed three times with 10% NaHCO₃ solution (each time 375 ml). The organic layer is again washed two times with water (each time 375 ml) and dried over Na₂SO₄. To the organic layer slowly added methyl tert-butyl ether hydrochloride solution at 25-30° C. for 30-45 minutes, the contents are stirred for 1 hour at 25-30° C., filtered the mass and washed with n-hexane. To the wet cake added diisopropyl ether (2 Lt) and 10% NaHCO₃ solution at 25-30° C., stirred for 45 minutes to 1 hour at 25-30° C. and then separated the layers. The organic layer is washed with water (1 Lt), dried over Na₂SO₄ and distilled off the solvent completely under vacuum at 45-50° C. To the residue added isopropyl alcohol (300 ml), heated to 60° C. and stirred for 20-30 minutes to form a clear solution. The resulting mass is slowly cooled to 5° C. in 45-50 minutes and stirred for 30-40 minutes at 5-10° C. Filtered the material, washed with chilled isopropyl alcohol (50 ml), dried under vacuum at 40° C. for 3-4 hours to give 100 gm of (2R,3S)-4-benzyl-3-(4-fluorophenyl)morpholinyl-3,5-bis(trifluoromethyl)benzoate.

Step-II: Dimethyl titanocene reagent Toluene (2.5 Lt) is added to titanocene dichloride (210 gm) under stirring at 25-30° C., the contents are cooled to 0° C. and then methyl magnesium chloride (1.25 Lt) is slowly added for 1 hour at 0-5° C. The resulting mass is stirred for 1 hour at 0-5° C., quenched the mass into the solution of NH₄Cl (200 gm) in water (3.25 Lt) at 10-15° C. under N₂ atmosphere and stirred for 20 minutes at 10-15° C. Separated the layers, the organic layer is washed three times with chilled water (each time 3.25 Lt) followed by saturated NaCl solution (3.25 Lt) and then dried over Na₂SO₄. Distilled off the solvent up to mass weight reaches to 1.6 Kg under vacuum at 40° C. to give dimethyl titanocene.

Step-III: (2R,3S)-4-Benzyl-2-[[1-[3,5-bis(trifluoromethyl)phenyl]vinyl]oxy]-3-(4-fluorophenyl)morpholine

(2R,3S)-4-Benzyl-3-(4-fluorophenyl)morpholinyl-3,5-bis(trifluoromethyl)benzoate (100 gm, obtained in step-I) is added to tetrahydrofuran under stirring at 25-30° C. followed by dimethyl titanocene (1.6 Kg, obtained in step-II) and titanocene dichloride (3 gm), the contents are heated to 70° C. and then stirred for 8 hours at 70- 75° C. The reaction mass is slowly cooled to 25-30° C. and then NaHCO₃ (39 gm), methanol (620 ml) and water (23.6 ml) are added. The contents are heated to 40-45° C., stirred for 5 hours at 40-45° C. and then cooled to 25-30° C. The reaction mass is stirred for over night at 25-30° C., filtered the salts and washed with n-hexane (500 ml). Distilled off solvent completely under vacuum at 40° C., co-distilled two times with methanol (each time 400 ml) and then stirred with methanol (760 ml) at 55-60° C. To the resulting mass added water (245 ml) slowly at 50-55° C. in 45-50 minutes, stirred for 15-20 minutes at 25-30° C. Cooled the mass to 20° C., stirred for 30 minutes, filtered the material and washed with the mixture of water and methanol (100 ml, 1:1) to give 90 gm of (2R,3S)-4-Benzyl-2-[[1-[3,5-bis(trifluoro-methyl)phenyl]vinyl]oxy]-3-(4-fluorophenyl)morpholine.

Step-IV: [2R-[2a(R*), 3a]]-2-[1-[3,5-bis(trifluoromethyl)-phenyl]ethoxy]-3-(4-fluorophenyl)morpholine p-toluene sulfonate salt

(2R,3S)-4-Benzyl-2-[[1-[3,5-bis(trifluoromethyl)phenyl]vinyl]oxy]-3-(4-fluorophenyl)morpholine (76 gm, obtained in step-III), ethyl acetate (930 ml), methanol (315 ml) and 10% Pd/C (19 gm) are taken into a hydrogenation flask at 25-30° C. and then subjected to hydrogenation by passing hydrogen gas under 40 psi pressure for 2 hours at 25-30° C. The reaction mass is filtered through hyflo bed, washed with ethyl acetate and the resulting filtrate is then subjected to carbon treatment. Distilled off solvent completely under vacuum, the residue is dissolved in methyl tert-butyl ether at 40° C. and then the solution of p-toluene sulfonic acid (26 gm) in methyl tert-butyl ether (135 ml) is added at 40° C. To the resulting mass added n-hexane (1.25 Lt) at 25-30° C. and stirred for 2 hours at 25-30° C. Filtered the material and washed with n-hexane to give 80 gm of crude [2R-[2a(R*),3a]]-2-[1-[3,5-bis(trifluoromethyl)-phenyl]ethoxy]-3-(4-fluorophenyl)morpholine p-toluene sulfonate salt (diastereomeric impurity: 17.2%).

The crude compound is added to toluene (600 ml), heated to 60-70° C. for 30 minutes to form a clear solution and then concentrated to half the initial volume by distilling off solvent at 40° C. The resulting mass is first cooled to 25-30° C. for 30 minutes and then to 10° C. for 1 hour. Filtered the material and washed with n-hexane to give 60 gm of pure [2R-[2a(R*),3a]]-2-[1-[3,5-bis(trifluoromethyl)-phenyl]ethoxy]-3-(4-fluorophenyl)morpholine p-toluene sulfonate salt (diastereomeric impurity: 1.6%).

Step-V: [2R-[2a(R*), 3a]]-5-[[2-[1-[3,5-bis(trifluoromethyl)phenyl]ethoxy]-3-(4-fluorophenyl)-4-morpholinly]methyl]-1,2-dihydro-3H-1,2,4-triazole-3-one, (or) 2-(R)-(1-(R)-(3,5-bis(trifluoro-methyl)-phenyl)ethoxy)-3-(S)-(4-fluoro)phenyl-4-(3-(5-oxo-1H,4H-1,2,4-triazolo)methylmorpholine); i.e. aprepitant

Potassium carbonate (10.7 gm) and dimethylsulfoxide (80 ml) are added to [2R-[2a(R*),3a]]-2-[1-[3,5-bis(trifluoromethyl)-phenyl]ethoxy]-3-(4-fluorophenyl) morpholine p-toluene sulfonate salt (19 gm, diastereomeric impurity: 1.6%) under N₂ atmosphere under stirring, the contents are cooled to 20° C. and then the solution of N-methylcarboxyl-2-chloroacetamidrazone (6 gm) in dimethylsulfoxide (77 ml) is slowly added during 30 minutes at 20-23° C. The contents are stirred for 1 hour at 20-23° C., the reaction mass is quenched into the mixture of water (150 ml) and methyl tert-butyl ether (300 ml) at 25-30° C. and then separated the layers. The organic layer is washed with water (230 ml) followed by washings with saturated sodium bicarbonate solution (230 ml), water (230 ml) and saturated sodium chloride solution (230 ml), dried over sodium sulfate and then concentrated at 40-50° C. The resulting residue is dissolved in xylene (250 ml), diisopropylethyl amine (62.5 ml) is added at 25-30° C., the contents are heated to 135° C. and then stirred for 3 hours. The reaction mass is cooled to 25-30° C. and stirred for over night at 25-30° C. Filtered the material and washed with xylene (10 ml) followed by n-hexane (100 ml) to give 6 gm of aprepitant (diastereomeric impurity: 1.1%).

### Reference Example 2

To a suspension of [2R-[2a(R*),3a]]-2-[1-[3,5-bis(trifluoromethyl)-phenyl]ethoxy]-3-(4-fluorophenyl)morpholine p-toluene sulfonate salt and powder K₂CO₃ in DMSO (7.8 Lt) at 20° C. is added a solution of N-methylcarboxyl-2-chloroacetamidrazone in DMSO(7.8 Lt). The first half of the solution is added quickly, (with slightly cooling with ice water bath) then the remaining half is added over a period of 1 hour. After the addition, the reaction is checked by LC, and the reaction is quenched with cold water (15 Lt) and methyl-t-butyl ether (MTBE) (30 Lt) solution. The organic layer is separated, and washed with water, sat. NaHCO₃, brine, and water (20 Lt/each) respectively. The aqueous layer is back extracted with additional MTBE (15 Lt). The combined MTBE solution is concentrated to an oil. The resulting crude product is dissolved in xylene (25 Lt) and diisopropylethylamine (6.25 Lt) and is heated to reflux (˜135° C.) and the reaction is monitored by LC. The reaction takes 4-6 hours to complete, the reaction solution is cooled down to room temperature overnight and filter to get [2R-[2a(R*),3a]]-5-[[2-[1-[3,5-bis(trifluoromethyl)phenyl]ethoxy]-3-(4-fluorophenyl)-morpholihly]methyl]-1,2-dihydro-3H-1,2,4-triazole-3-one (expect 1.33 Kg, ˜80%, typically purity 98.5A %). The resulting crude product is dissolved in hot methanol (13.3 Lt), added charcoal 133 gm, then filtered and the charcoal is washed with hot methanol (3.3 Lt). The methanol solution is cooled down to room temperature, then water (7 Lt) is added drop wise. After being stirred at room temperature for 2 hrs, the suspension is filtered to pure aprepitant as a white crystalline compound (Purity, 99.5%, diastereomeric impurity: 0.4%).

### Example 1

Aprepitant (5 gm; obtained by the process described in reference example 1, HPLC purity:98.5%, content of diastereomeric impurity: 1.1%) is dissolved in ethyl acetate (100 ml) at 70° C., stirred for 30 minutes and then distilled off ethyl acetate under atmospheric conditions until the collected volume reaches to 50 ml. The reaction mass is gradually cooled to 25-30° C. and then to 0-5° C., and stirred for 1 hour. Filtered the solid, washed with chilled ethyl acetate (10 ml) and then dried at 60° C. to give 4 gm of pure aprepitant (HPLC purity: 99.97%, diastereomeric impurity: Not detected, mean particle size: 4.95 microns).

### Example 2

Potassium carbonate (10.7 gm) and dimethylsulfoxide (80 ml) are added to [2R-[2a(R*),3a]]-2-[1-[3,5-bis(trifluoromethyl)-phenyl]ethoxy]-3-(4-fluorophenyl) morpholine p-toluene sulfonate salt (19 gm, diastereomeric impurity: 17-18%) under N₂ atmosphere under stirring, the contents are cooled to 20° C. and then the solution of N-methylcarboxyl-2-chloroacetamidrazone (6 gm) in dimethylsulfoxide (77 ml) is slowly added during 30 minutes at 20-23° C. The contents are stirred for 1 hour at 20-23° C., the reaction mass is quenched into the mixture of water (150 ml) and methyl tert-butyl ether (300 ml) at 25-30° C. and then separated the layers. The organic layer is washed with water (230 ml) followed by washings with saturated sodium bicarbonate solution (230 ml), water (230 ml) and saturated sodium chloride solution (230 ml), dried over sodium sulfate and then concentrated at 40-50° C. The resulting residue is dissolved in xylene (250 ml), diisopropylethyl amine (62.5 ml) is added at 25-30° C., the contents are heated to 135° C. and then stirred for 3 hours. The reaction mass is cooled to 25-30° C. and stirred for over night at 25-30° C. Filtered the material and washed with xylene (10 ml) followed by n-hexane (100 ml) to give 6 gm of aprepitant (diastereomeric impurity: 13%)

The aprepitant (6 gm, obtained above) is added to ethyl acetate (120 ml) at 25-30° C., heated to 70° C. to form a clear solution and then stirred for 15 minutes. The reaction mass is then subjected to carbon treatment at 70° C., washed the bed with hot ethyl acetate (10 ml) and the resulting filtrate is concentrated until the solvent volume reaches to 70 ml without vacuum. The resulting mass is slowly cooled to 0-5° C., stirred for 1 hour, filtered the solid and washed with chilled ethyl acetate (10 ml) to give 4 gm of aprepitant (diastereomeric impurity: 1.5%).

The aprepitant obtained above is stirred with ethyl acetate (60 ml) at 70° C. to form a clear solution, distilled off the solvent without vacuum until the solvent volume reaches to 30 ml, cooled slowly to 0-5° C. and then stirred for 1 hour. Filtered the solid, washed with 5 ml of chilled ethyl acetate and dried to give 3 gm of pure aprepitant (diastereomeric impurity: 0.04%).

### Example 3

Aprepitant (2 gm) is dissolved in methanol (25 ml) at 55-60° C., distilled off the solvent completely and then water (25 ml) is added. The contents are stirred at 25-30° C. for 1 hour, filtered the solid, washed with water and then dried under vacuum for 5 hours at 40° C. to give 1.82 gm of aprepitant crystalline form II.

### Example 4

Aprepitant (2 gm) is dissolved in methanol (50 ml) at 25-30° C. and then water (25 ml) is added. The contents are stirred for 30 minutes at 25-30° C., filtered the solid, washed with water and then dried under vacuum for 5 hours at 40° C. to give 1.84 gm of aprepitant crystalline form II.

### Example 5

Crystalline aprepitant (10 gm) is dissolved in methanol (250 ml) at 25-30° C. and the solution is subjected to spray drying at 80° C. for 2 hours to give amorphous aprepitant.

### Example 6

Crystalline aprepitant (10 gm) is dissolved in acetone (350 ml) at 25-30° C. and the solution is subjected to spray drying at 70° C. for 2 hours 30 minutes to give amorphous aprepitant.

### Example 7

Crystalline aprepitant (5 gm) is dissolved in ethyl acetate (100 ml) at 70° C. and then stirred for 30 minutes. The solution is cooled to 25-35° C. and then subjected to spray drying at 95° C. for 1 hour 30 minutes to give amorphous aprepitant.

1. A process for preparing crystalline form II aprepitant which comprises:
a) distilling off a solvent from a solution of aprepitant in a solvent selected from the group consisting of methanol, ethanol, isopropyl alcohols and tert-butyl alcohol at least until precipitation of aprepitant occurs;
b) separating the solid aprepitant, if necessary;
c) slurrying the solid aprepitant in water; and
d) separating the crystalline form II aprepitant from the contents.

2. The process as claimed in claim 1, wherein the distillation of the solvent in step (a) is carried out at atmospheric pressure or at reduced pressure.

3. The process as claimed in claim 1, wherein the distillation of the solvent in step (a) is carried out just until precipitation of aprepitant starts forming.

4. The process as claimed in claim 1, wherein the distillation of the solvent in step (a) is carried out until substantial precipitation occurs.

5. The process as claimed in claim 1, wherein the distillation of the solvent in step (a) is carried out until the solvent is almost completely distilled off.

6. The process as claimed in claim 1, wherein the separation of the precipitated solid aprepitant in step (b) is carried out by filtration or centrifugation.

7. The process as claimed in claim 1, wherein the slurring in step (c) is carried out at about 20° C. to 80° C.

8. The process as claimed in claim 1, wherein the crystalline form II of aprepitant is collected from the slurry in step (d) by filtration or centrifugation.

9. A process for preparation of aprepitant substantially free of diastereomeric impurity, namely 5-[2(S)-[1 (RS)-[3,5-bis(trifluoromethyl)-phenyl)ethoxy]-3-(S)-(4-fluorophenyl)-morpholin-4-yl-methyl]-3,4-dihydro-2H-1,2,4-triazol-3-one, which comprises crystallizing aprepitant substantially free of diastereomeric impurity from a solution of aprepitant contaminated with the diastereomeric impurity in ethyl acetate.

10. The process as claimed in claim 9, wherein the aprepitant obtained has a content of diastereomeric impurity of less than about 0.1% by weight.

11. The process as claimed in claim 10, wherein the aprepitant has a diastereomeric impurity of less than about 0.05% by weight.

12. The process as claimed in claim 11, wherein the aprepitant contains no diastereomeric impurity.

13. A process for purification of aprepitant, which comprises crystallizing aprepitant from a solution of crude aprepitant in ethyl acetate.

14. The process as claimed in claim 13, wherein the crude aprepitant is dissolved in ethyl acetate at an elevated temperature.

15. The process as claimed in claim 13, wherein the crystallization of the aprepitant is initiated by cooling, seeding, partial removal of the solvent from the solution, by adding an anti-solvent to the solution or a combination thereof.

16. Aprepitant having a mean particle size of less than about 11.5 microns.

17. A process for the preparation of the aprepitant having a mean particle size of less than about 11.5 microns of claim 16, which comprises crystallizing aprepitant having mean particle size of less than about 11.5 microns from a solution of aprepitant in ethyl acetate.

18. A process as claimed in claim 17, wherein the aprepitant is dissolved in ethyl acetate at an elevated temperature.

19. The process as claimed in claim 17, wherein the crystallization of the aprepitant is initiated by cooling, seeding, partial removal of the solvent from the solution, by adding an anti-solvent to the solution or a combination thereof.

20. The compound as claimed in claim 16, wherein the aprepitant has a mean particle size between about 2-10 microns.

21. The compound as claimed in claim 20, wherein the aprepitant has a mean particle size between about 3-8 microns.

22. Amorphous aprepitant.

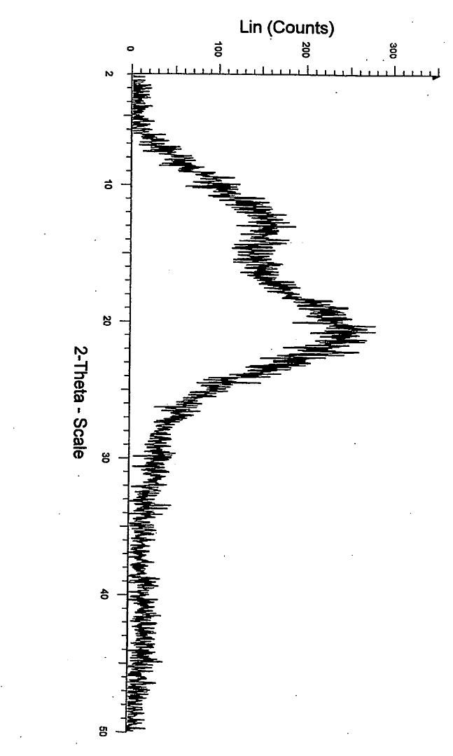

23. The compound as claimed in claim 22, wherein the amorphous aprepitant is characterized by an X-ray powder diffraction spectrum as shown in FIG. 1.

24. The process for preparation of amorphous aprepitant as defined in claim 22, which comprises dissolving aprepitant in an alcoholic solvent, a ketonic solvent or an ester solvent, and then removing the solvent from the solution by spray drying or freeze drying.

25. The process as claimed in claim 24, wherein the alcoholic solvent is selected from the group consisting of methanol, ethanol, isopropyl alcohol, tert-butylalcohol and n-butyl alcohol; the ketonic solvent is selected from the group consisting of acetone, diethyl ketone, methyl ethyl ketone, methyl isobutyl ketone and methyl propyl ketone; and the ester solvent is selected from ethyl acetate, methyl acetate and isobutyl acetate.

26. The process as claimed in claim 25, wherein the alcoholic solvent is methanol.

27. A pharmaceutical composition comprising the aprepitant having a mean particle size of less than about 11.5 microns of claim 16 and a pharmaceutically acceptable excipient.

28. The pharmaceutical composition as claimed in claim 27, wherein the pharmaceutical composition of aprepitant having mean particle size of less than about 11.5 microns is a solid oral dosage form.

29. A pharmaceutical composition comprising the amorphous aprepitant of claim 22 and a pharmaceutically acceptable excipient.

30. The pharmaceutical composition as claimed in claim 29, wherein the pharmaceutical composition of amorphous aprepitant is a solid oral dosage form.
